(19) United States
(12) Patent Application Publication
(10) Pub. No.: US 2025/0195751 A1
(43) Pub. Date: Jun. 19, 2025

(54) SYSTEM FOR CONTROLLING A PRODUCT ADMINISTRATION DEVICE, AND ASSOCIATED PRODUCT ADMINISTRATION DEVICE

(71) Applicant: Nemera La Verpillière, La Verpilliere (FR)

(72) Inventor: Sébastien DELVALAC, Lyon (FR)

(73) Assignee: Nemera La Verpillière, La Verpilliere (FR)

(21) Appl. No.: 19/072,627

(22) Filed: Mar. 6, 2025

Related U.S. Application Data
(63) Continuation of application No. PCT/EP2023/074538, filed on Sep. 7, 2023.

(30) Foreign Application Priority Data
Sep. 8, 2022 (FR) FR2209013

(51) Int. Cl. A61M 5/142; A61M 5/158

(52) U.S. Cl. CPC A61M 5/14236; A61M 5/158; A61M 2005/1585

(57) ABSTRACT

This control system for controlling a product administering device comprises a drive assembly, a piston, a control member and an activation element for activating a device for inserting a needle into a site. The control member comprises a threaded rod intended to be rotationally driven by the drive assembly and a driving element intended to be translationally driven along the threaded rod when the threaded rod is rotationally driven, to cause the piston to move. The activation element is movable between a rest position and an activation position for activating the needle inserting device. The driving element is also capable of driving the movement of the activation element between its rest position and its activation position as it is translationally driven along the threaded rod. The activation element is translationally movable between its rest position and its activation position.

The present invention relates to a system for controlling a device for administering a product, in particular a drug intended to be administered parenterally. The invention also relates to a device for administering a product and comprising the control system.

In the field of administering products and in particular drugs parenterally, it is known practice to use portable injection devices intended to be fixed to a patient's body, in particular by means of an adhesive patch, to perform an infusion of the drug, i.e. intradermally, intramuscularly, subcutaneously, etc. Such devices are advantageous for a patient in that they can have the drug administered parenterally at home, over a relatively lengthy duration, which can range up to several hours, without having to hold themselves still. Furthermore, these devices advantageously make it possible to administer a drug without the assistance of caregiving staff.

Advantageously, some of these injection devices comprise a disposable part provided with a container of product and with a needle insertion device, and a reusable part which is able to be fixed to the disposable part and comprises for example a reusable control system for causing the product to be discharged from the container.

In particular, WO2017/219156 A1 discloses a system for controlling a product administering device, and an associated product administering device. The product administering device is intended to be fixed to a patient's body, in particular by means of an adhesive patch, and comprises the control system, a container for a drug, and a device for inserting a needle into a site. In WO2017/219156 A1, the system for controlling the administering device makes it possible to cause both the product to be discharged from the container of product and a device for inserting a needle into a site to be activated. However, the control system is mechanically complex, which in particular results in a high cost and a high risk of failure.

Additionally, US2020/0114068 discloses mounting a nut on a threaded rod of a system for controlling a product administering device and causing a lever to tilt in rotation, which makes it possible to activate a needle insertion device. The rotation of the lever takes up space, meaning that this control system is bulky and cannot be conveniently integrated in a product administering device, such as a portable drug administering device.

Other systems known from WO2016/145094, WO2022/104362, EP3260146, EP3501576 and WO2018/204779 give rise to drawbacks comparable to those mentioned above.

An aim of the invention is therefore to provide a system for controlling a product administering device which has a simpler structure and better reliability.

To that end, the invention relates to a control system for controlling a product administering device, the control system comprising a drive assembly, a piston, a control member and an activation element for activating a device for inserting a needle into a site. The control member comprises a threaded rod intended to be rotationally driven by the drive assembly, and a driving element translationally driven along the threaded rod when the threaded rod is rotationally driven, to cause the piston to move. The activation element is movable between a rest position and an activation position for activating the needle inserting device. The driving element also drives the movement of the activation element between its rest position and its activation position as it is translationally driven along the threaded rod. In accordance with the invention, the activation element is translationally movable between its rest position and its activation position.

By virtue of the invention, the movement of the driving element along the threaded rod makes it possible to both discharge the product from the container of product and activate the needle inserting device. The structure of the control system is thus simplified and the reliability of the system is improved because the movement of the driving element, which is essential to administering the product, is also used to activate the needle inserting device.

According to various aspects of the invention, the control system comprises one or more of the following features, considered alone or in any technically permissible combinations:
the driving element drives the translational movement of the activation element from its rest position to its activation position and the translational movement of the activation element from its activation position to its rest position;
the piston comprises a flexible rod and a thrust element able to act on a piston plunger of a container of product to discharge the product from the container, the flexible rod comprising a driving end intended to be in contact with the driving element and a thrust end intended to be in contact with the thrust element;
the flexible rod is formed by a spring and the activation element is fixed to the threaded rod, the spring being capable of exerting a thrust force on the threaded rod via the driving element, so as to move the activation element from its rest position to its activation position when the thrust element pushes against the piston plunger and when the driving element is translationally driven along the threaded rod in a direction of deployment of the piston;
the driving end is fixed to the driving element and the thrust end is fixed to the thrust element, and the spring is capable of exerting a pulling force on the threaded rod via the driving element, so as to move the activation element from its activation position to its rest position when the driving element is translationally driven along the threaded rod in a direction of retraction of the piston;
the driving element is capable of moving the piston to cause the product to be discharged from the container, when the driving element is translationally driven along the threaded rod in a direction of deployment of the piston, and of moving the activation element from its rest position to its activation position, when the driving element is translationally driven along the threaded rod in a direction of retraction of the piston;
the system comprises a return element capable of exerting a return force on the activation element for returning it to its rest position;
the activation element comprises a rod around which extends the return element and a shoulder in contact with the return element;
the activation element is received in a region comprising a first end and a second end which are opposite one another along an axis of movement of the activation element, the first end defining a stop in contact with the activation element when it is in its rest position;
the second end defines either a second stop in contact with the activation element, when it is in its activation position, or a bearing surface for a return element;
the system comprises a first casing for receiving the drive assembly, the control member and the activation element, and the first casing comprises a first orifice for the passage of the activation element and through which the activation element can pass when it is in its activation position, the activation element then protruding from the first casing;
the system comprises a first casing for receiving the drive assembly, the control member and the activation element, and the first casing is capable of being assembled detachably with a second casing which receives the needle inserting device and advantageously a container of product;
the system comprises a first cavity capable of receiving the flexible rod, the threaded rod and the driving element, the first cavity comprising a wall provided with a longitudinal slot, and the driving element comprises a radial protrusion intended to be received in the longitudinal slot, so as to prevent the driving element from rotating relative to the first cavity; and
the activation element comprises a contact end and an activation end, the contact end defining a cavity for receiving the radial protrusion, when the driving element drives the activation element from its rest position to its activation position via the radial protrusion.

The invention also relates to a product administering device comprising a first part provided with a control system as mentioned above and a second part which receives the needle inserting device and a container of product.

Advantageously, the control system comprises a first casing for receiving the drive assembly, the control member and the activation element, and the first casing comprises a first orifice for the passage of the activation element and through which the activation element can pass when it is in its activation position, the activation element then protruding from the first casing, whereas the second part comprises a second casing for receiving the needle inserting device, the second casing being intended to be assembled detachably with the first casing, and the second casing comprises a wall provided with a second orifice capable of being positioned facing the first orifice when the first casing and the second casing are assembled, the activation element being capable, when it is in its activation position, of passing through the second orifice to activate the needle inserting device and trigger the insertion of the needle into a site.

### BRIEF DESCRIPTION OF THE FIGURES

The invention will be better understood and further advantages thereof will become apparent in the light of the following description, which is given solely by way of nonlimiting example with reference to the appended drawings, in which:

FIG. 1 is a perspective view of a product administering device according to a first embodiment of the invention, comprising a reusable control system in accordance with the first embodiment of the invention, the administering device being ready for use;

FIG. 2 is a perspective view of the assembling of a disposable part on the reusable control system of the device in FIG. 1;

FIG. 3 is a perspective view of the reusable control system in FIGS. 1 and 2, with one half-shell of a casing omitted;

FIG. 4 is an exploded view of the reusable control system in FIG. 3;

FIG. 5 is an exploded view, on a larger scale, of the detail V in FIG. 4;

FIG. 6 is a partial top view of the product administering device in FIGS. 1 to 2 comprising the reusable control system in FIGS. 3 and 4, with one half-shell of a casing omitted, and a container of product associated with said control system, with the control system in a rest configuration;

FIG. 7 is a view similar to FIG. 6, with the control system in an activation position for activating a device for inserting a needle into a site;

FIG. 8 is a view similar to FIGS. 6 and 7, with the control system in a configuration for bringing a thrust element into contact with a piston plunger;

FIG. 9 is a view similar to FIGS. 6 to 8, with the control system in a configuration where the product has finished being discharged from the container;

FIG. 10 is a partial top view of a product administering device according to a second embodiment comprising a reusable control system according to the second embodiment of the invention, with one half-shell of a casing omitted, and a container of product associated with said control system, with the control system in a rest configuration;

FIG. 11 is a view similar to FIG. 10, with the control system in a configuration for bringing a thrust element into contact with a piston plunger;

FIG. 12 is a view similar to FIGS. 10 and 11, with the control system in an activation position for activating a device for inserting a needle into a site;

FIG. 13 is a view similar to FIGS. 10 to 12, with the control system in a configuration where the product has finished being discharged from the container.

### DETAILED DESCRIPTION

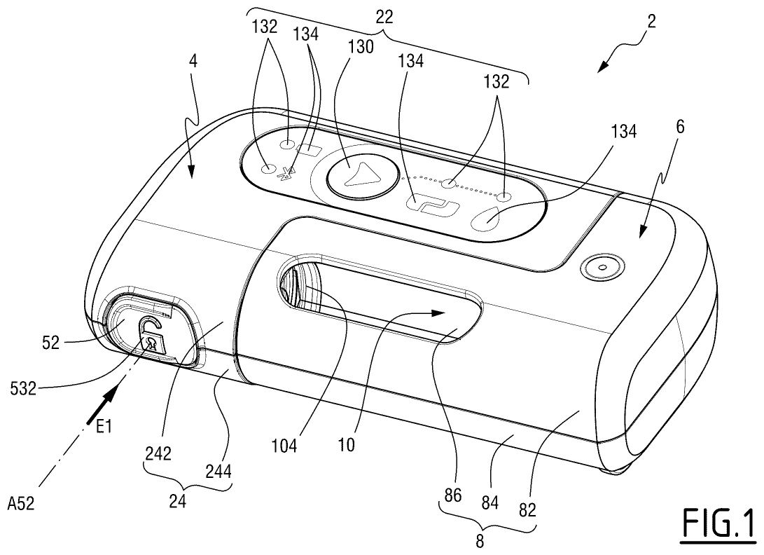

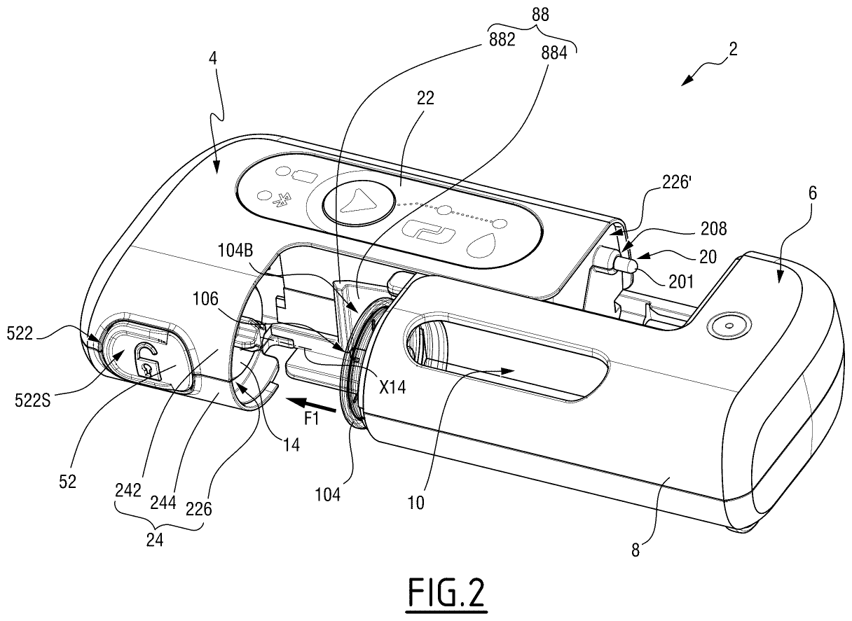

The product administering device 2 depicted in its entirety in FIGS. 1 and 2 is in accordance with a first embodiment of the invention and comprises a reusable control system 4 according to the first embodiment of the invention, and a disposable part 6.

This disposable part 6 comprises a casing 8 formed of two half-shells 82 and 84 which enclose a cartridge 10 of product that is to be administered, this cartridge being partially visible through a cut-out window 86 in the half-shell 82 of the casing 8.

The cartridge 10, also referred to as container of product, comprises a body 102, made of glass or of plastics material, and a sliding plunger 104, also referred to as “piston plunger”, which is preferably made of elastomer and is designed to slide inside the cartridge along a longitudinal axis X10 of the cartridge 10.

In the figures, the cartridge 10 is depicted as see-through.

The cartridge 10 is intended to be connected, at the opposite end from the sliding plunger 104, to a product administering subassembly, not depicted, which comprises in particular a device for inserting a needle into a site and a fluidic path extending between the cartridge and the needle inserting device. The needle inserting device is, for example, provided with a needle and a cannula and comprises a control assembly for controlling the position of the needle and of the cannula. The control assembly is, for example, capable of moving the needle and the cannula from a retracted position in which they are contained inside the casing 8 to a deployed position in which the needle and the cannula extend out of the casing 8 and are capable of being positioned through a user's skin. The control assembly is also capable, after the needle has been inserted through the user's skin, of moving the needle from the deployed position to the retracted position in order to keep only the cannula in the user's skin, with the product delivered to the user via said cannula. The needle inserting device is, for example, in accordance with the technical teaching of either of the documents WO-A-2021/224388 and WO-A-2022/023391. Advantageously, the needle inserting device comprises a release member for releasing the needle and the cannula, the release member being capable, when it is moved via an activation element, of permitting the movement of the needle and of the cannula from the retracted position to the deployed position.

Advantageously, the disposable part 6 also comprises the administering subassembly received in the casing 8.

The part 6 is intended to be mounted on the control system 4 to slide in the direction of the arrow F1 in FIG. 2, to be controlled by the control system 4 at the time of administering the product contained in the cartridge 10 to a user, and then be disposed of when the cartridge 10 has been emptied or when the quantity of product that is to be administered that is present in this cartridge reaches a predefined minimum value. Thus, multiple disposable parts 6 may be mounted in succession on the control system 4 which is reused with each of these different parts 6.

The control system 4 comprises a drive assembly 12, a piston 14, a control member 16, an activation element 20 for activating the needle inserting device, and advantageously a control interface 22.

The control system 4 advantageously comprises a casing 24 formed of two half-shells 242 and 244. As depicted in FIGS. 1 to 3, the piston 14, the control member 16 and the activation element 20 are accommodated in the casing 24 and the control interface 22 is positioned on an outer face of the casing 24, for example on the half-shell 242.

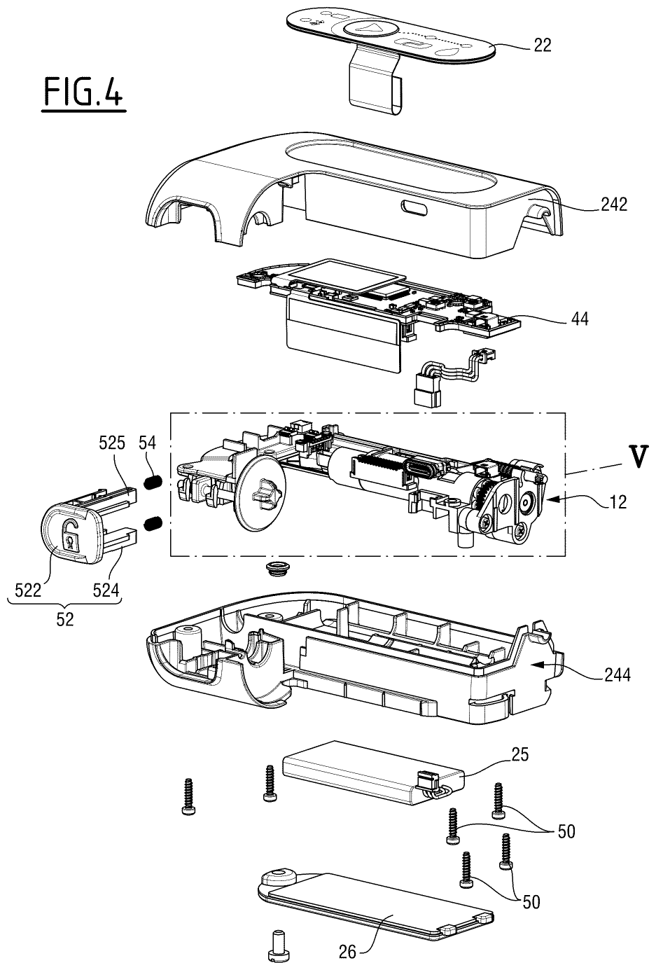

The control system 4 also comprises a battery 25, depicted in FIG. 4 and held in place in the half-shell 244 by a cover 26, which is attached to and held securely on this half-shell.

The drive assembly 12 comprises an electric motor 42 and at least one gearset 46, which is driven by the motor 42.

The motor 42 is able to selectively rotationally drive the gearset 46 in a first direction and an opposite second direction to the first direction.

The piston 14 is configured to push the sliding plunger 104 toward a product discharge end of the cartridge 10, along the axis X10, so as to cause the product to be discharged. This piston 14 is visible via a lateral opening formed on a lateral face 226 of the casing 24. The movement of the piston for pushing the sliding plunger 104 causes the product contained in the cartridge 10 to be discharged out of the cartridge 10.

The piston 14 comprises a piston rod 141 and a thrust element 143 fixed to the piston rod.

The thrust element 143 is capable of being positioned facing the sliding plunger 104 when the casing 8 and the casing 24 are assembled one with the other. The thrust element 143 is visible via the lateral opening formed on the lateral face 226 when the casing 24 is disassembled from the casing 8.

A front face 143A of the thrust element 143 is defined as being that one of its faces that faces toward the plunger 104 when the parts 4 and 6 are assembled to form the device 2. A rear face 143B of the thrust element 143 is defined as being a face that faces in the opposite direction from the front face.

The piston rod 141 comprises a driving end 141A intended to be in contact with the control member 16 and a thrust end 141B intended to be in contact with the thrust element 143.

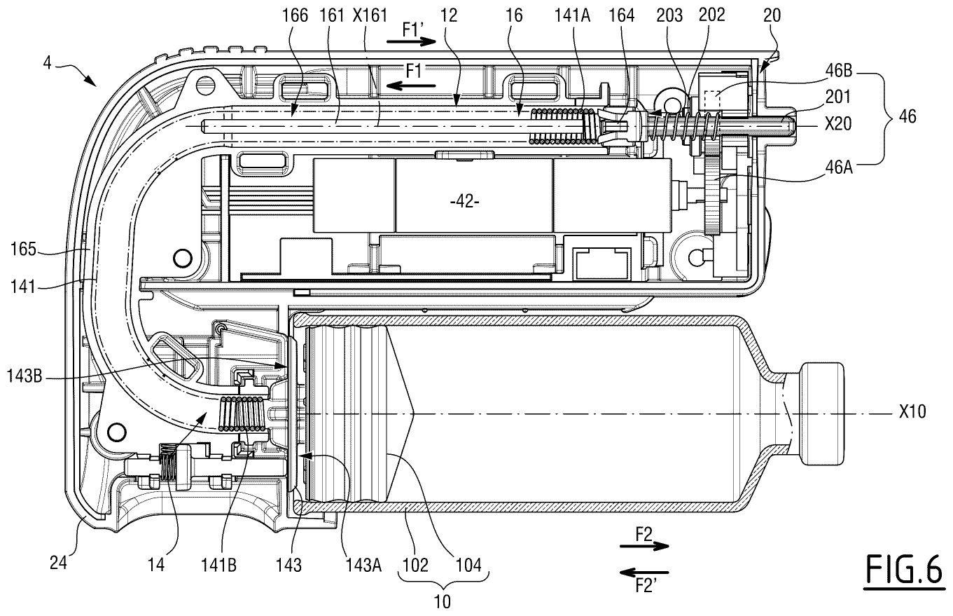

In FIG. 6 et seq., the piston rod 141 is depicted in a centerline line style between its ends 141A and 141B.

The piston rod 141 is, for example, a flexible rod formed by a coil spring, and the thrust element 143 is capable of being fixed to the spring for example by screwing it onto the thrust end 141B. The turns of the spring form, for example, at the end 141B, a tapping able to collaborate with a corresponding screw thread formed on a stud provided on the rear face 143B of the thrust element 143, in an attachment region 145 for attaching the thrust element 143.

The thrust element 143 is capable of acting on the piston plunger to discharge the product from the container 10. The thrust element 143 is capable of passing through the lateral opening so as to come into contact with the piston plunger 104 on discharge of the product from the container.

The front face 143A of the thrust element 143 is equipped with a protuberance, in other words a tip, 146 intended to enter a correspondingly shaped cavity 106 provided on a rear side 104B of the plunger 104 that faces toward the piston 14 when the product administering device 2 is in the configuration for use.

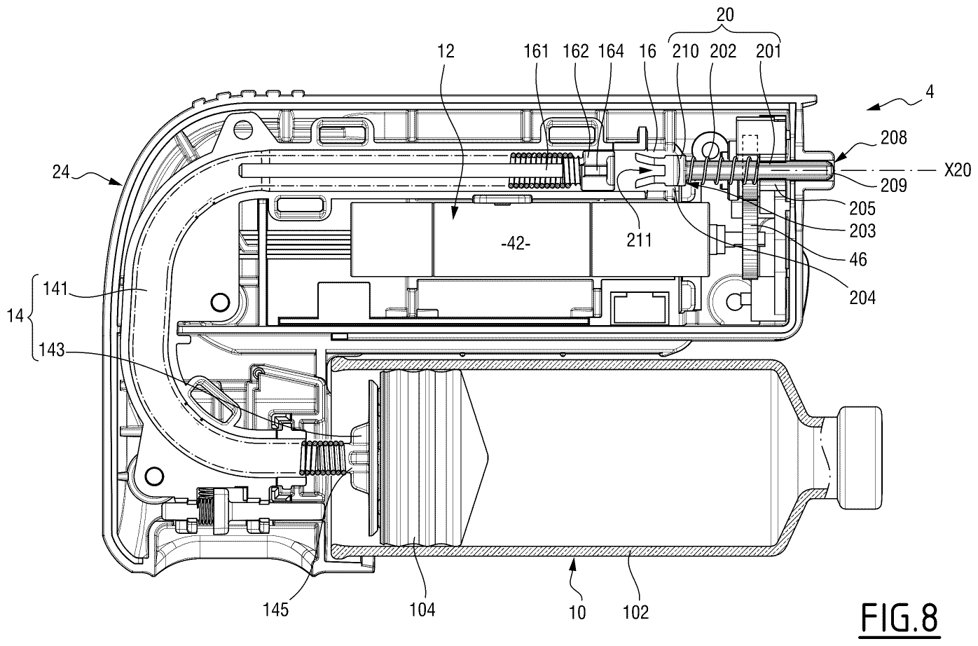

The thrust element 143 is movable between a rest position and multiple operating positions. In the rest position, depicted particularly in FIG. 6, the thrust element 143 presses against the casing 24 and is advantageously disengaged from the body 102 of the cartridge 10. In one of the operating positions, as presented in FIG. 8, the thrust element 143 pushes the plunger 104 toward the product discharge end of the cartridge 10 and is advantageously engaged in the body 102. Multiple operating positions of the piston 14 are distributed along an axis X14 of movement of the thrust element, which coincides with the axis X10 when the product administering device 2 is in the assembled configuration.

The control member 16 comprises a threaded rod 161 intended to be rotationally driven by the drive assembly 12, and a driving element 162 intended to be translationally driven along the threaded rod 161 when the threaded rod 161 is rotationally driven, to cause the piston 14 to move. The movements of the driving element 162 are parallel to the longitudinal axis X161 of the threaded rod 161.

The threaded rod 161 is for example associated with the gearset 46 which is rotationally driven by the electric motor 42. The gearset 46 comprises for example a first toothed wheel 46A and a second toothed wheel 46B which mesh with one another, with the second toothed wheel 46B fixed to the threaded rod 161 and capable of being rotationally driven by the electric motor 42 via the first toothed wheel 46A.

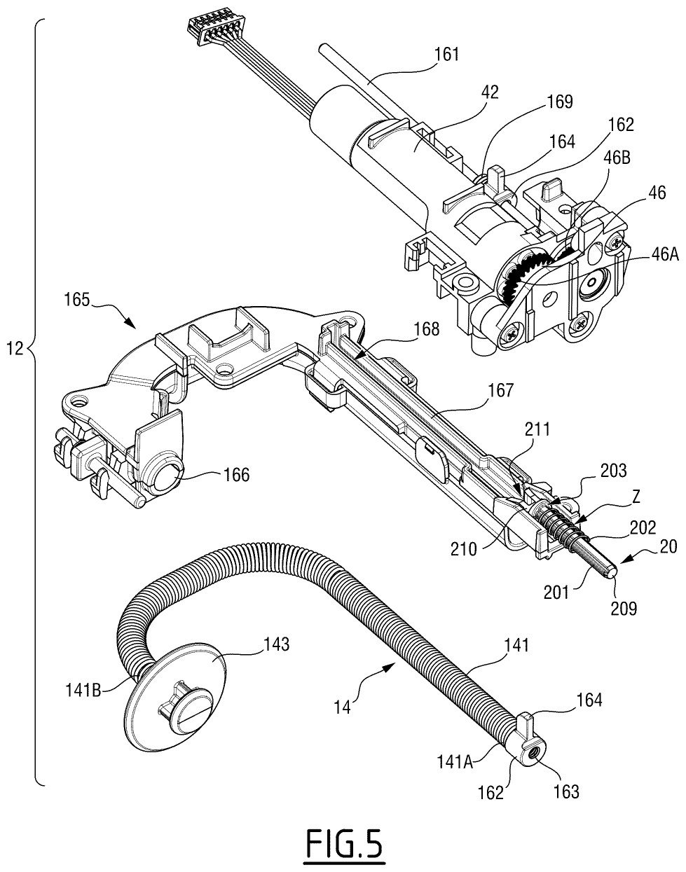

The driving element 162 is fixed to the driving end 141A of the piston rod, which is the opposite end from the thrust end, and is able to cause the piston rod 141 to move, and thus, during this movement, the thrust element 143 to move. The driving element 162 is for example capable of being fixed to the spring that forms the piston rod 141 by screwing at the driving end 141A. The driving element 162 is, for example, formed by a nut, shown in FIG. 5, which comprises a radial protrusion 164 and an external thread 169 able to collaborate with the turns of the spring at the driving end 141A which form a complementary tapping for fixing the driving element 162 to the piston rod 141. The driving element 162 is provided with an internal tapping 163 provided to collaborate with the thread of the threaded rod 161.

In FIG. 5, the parts 162 and 164 of the drive assembly 12 are depicted twice, in order to show their positioning both with respect to the threaded rod 161 and with respect to the piston 14.

The radial protrusion 164 is able to interact with the activation element 20 to cause the latter to move.

Advantageously, the control member 16 also comprises a channel 165 which is configured in the overall shape of a J and is capable of receiving the piston rod 141. As a result, the channel gives the piston rod a curved shape when it is positioned therein such that the piston rod comprises a first rectilinear part at its driving end 141A, a second rectilinear part at its thrust end 141B, and a curved part connecting the first rectilinear part and the second rectilinear part.

With preference, the channel forms a first cavity 166 capable of receiving the piston rod 141, the threaded rod 161 and the driving element 162. The first cavity 166 comprises a wall 167 provided with a longitudinal slot 168 in which the radial protrusion 164 of the driving element 162 is received so as to prevent the driving element from rotating relative to the first cavity and thus prevent the driving element from rotating as the threaded rod 161 rotates. The channel 165 is, for example, formed by two half-shells, i.e. a lower half-shell and an upper half-shell assembled with one another.

The threaded rod 161 and the driving element 162 form a mechanism for converting the rotational movement output by the gearset 46 into a translational movement intended to be transmitted to the piston 14 and particularly to the thrust element 143 along the axis X14 when the product administering device 2 is in the assembled configuration.

In this case, the connection between the elements 161 and 162 is of the screw-nut type.

The driving element 162 moved by the threaded rod 161 is thus capable of driving the movement of the piston 14, by acting on the driving end 141A of the piston rod 141, to in particular cause the piston plunger 104 to move within the cartridge 10 and thus the product to be discharged from the cartridge 10.

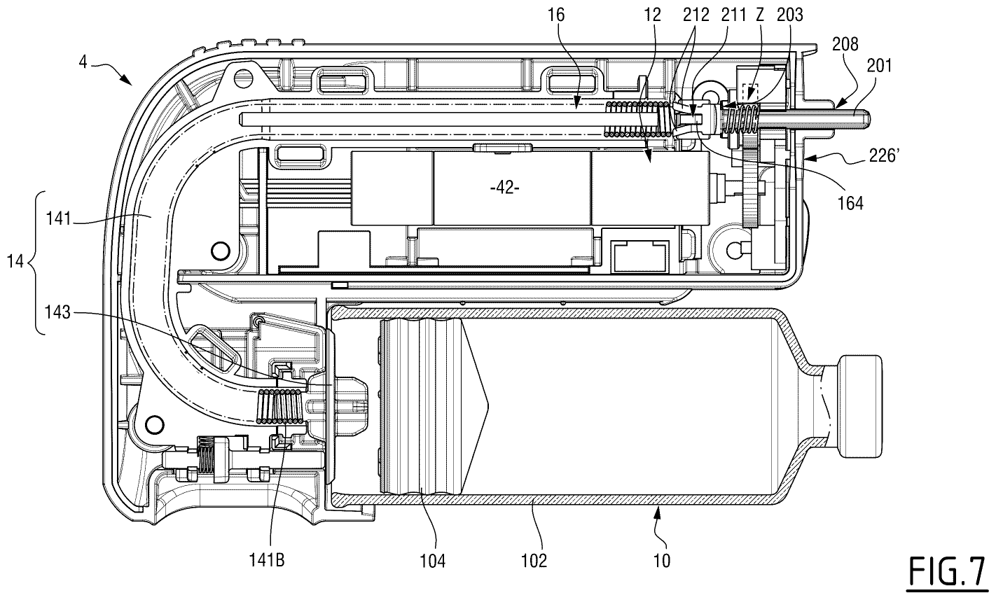

The driving element 162 is also capable, while being translationally driven along the threaded rod 161, of driving the translational movement of the activation element 20 between a rest position, shown in FIG. 6, and an activation position for activating the needle inserting device, that position being shown in FIG. 7.

More specifically, in the first embodiment depicted in FIGS. 1 to 9, when the motor 42 rotationally drives the gearset 46 in the first direction, the driving element 162 is translationally driven along the threaded rod in a direction of deployment of the piston 14 to cause the product to be discharged from the container. In other words, the driving element 162 is translationally driven in order to move the thrust element 143 of the piston 14 toward the discharge end of the cartridge 10, to discharge product from the cartridge 10. Thus, owing to the curved shape of the piston rod 141, when the driving element 162 is moved in the piston deployment direction, the thrust element 143 is moved in a direction corresponding to the arrow F2 marked in FIG. 6, whereas the driving element 162 is moved in an opposite direction corresponding to the direction of the arrow F1 marked in FIG. 2 and reproduced in FIG. 6.

Moreover, when the motor 42 rotationally drives the gearset 46 in the second direction, the driving element 162 is translationally driven along the threaded rod in a direction of retraction of the piston 14, in the direction of the arrow F1′ marked in FIG. 6, so as to cause the piston to retract and the activation element 20 to move between the rest position and the activation position for activating the needle insertion device, when the driving element 162, and particularly the radial protrusion 164, are in contact with the activation element 20. In other words, the driving element 162 is translationally driven in order to move the piston 14 toward the casing 24 away from the product discharge end of the cartridge 10, in the direction of the arrow F2′ marked in FIG. 6. Thus, owing to the curved shape of the piston rod 141, when the driving element is moved in the direction of retraction of the piston 14, the thrust element 143 is moved in a direction corresponding to the arrow F2′ whereas the driving element 162 is moved in an opposite direction, corresponding to the direction of the arrow F2 marked in FIG. 6.

Advantageously, the activation element 20 is capable, while being translationally moved from its rest position to its activation position, of coming into contact with the release member and actuating it in order to permit the needle and the cannula to move from their retracted position to their deployed position.

The activation element 20 comprises for example a rod 201 for selectively activating the needle inserting device.

More specifically, in the first embodiment, the activation element 20 comprises the rod 201 around which extends a return element 202 and a shoulder 203, extending radially to the rod 201 and in contact with the return element 202. The return element 202 is capable of exerting a return force on the activation element 20 for returning it to its rest position.

In greater detail, the activation element 20 is received in a receiving region Z of the casing 24 that comprises a first end 204 and a second end 205 which are opposite one another along an axis of movement X20 of the activation element 20. The first end 204 defines a stop in contact with the activation element 20 when it is in its rest position and the second end 205 defines a bearing surface for the return element 202. The return element thus extends between the second end 205 and the shoulder 203. The receiving region Z is, for example, formed on an upper face of the channel 165, as shown in FIG. 5.

Advantageously, the return element 202 is a compression spring.

In the rest position of the activation element 20, the rod 201 is kept inside the casing 24 and in its activation position the rod 201 protrudes from the casing 24, particularly from a lateral face 226′ parallel to the lateral face 226, advantageously through a first orifice 208 for passage of the activation element, this orifice being formed through a wall of the casing 24.

Advantageously, the casing 8 comprises a wall provided with a second orifice, not depicted, which is capable of being positioned facing the first orifice 208 when the casing 24 and the casing 8 are assembled. The activation element 20 is capable, when it is in its activation position, of passing through the first orifice 208 and the second orifice to activate the needle inserting device and trigger the insertion of the needle into a site.

Advantageously, the activation element 20 comprises an activation end 209 formed for example by a free end of the rod 201 away from the shoulder 203 and a contact end 210 extending away from the rod and into the casing 24. The contact end 210 defines a cavity 211 for receiving the radial protrusion 164, when the driving element 162 drives the activation element 20 from its rest position to its activation position via the radial protrusion 164. The contact end 210 comprises, for example, two arms 212 extending from the shoulder 203 into the casing 24 and defining a slot for receiving the radial protrusion 164. The activation element 20 is driven by the driving element 162 mechanically, and this is the result of the radial protrusion 164 being brought to bear in the cavity 211 of the contact end 210.

Advantageously, the translational movements of the activation element 20 take place in two opposite directions, between its rest and activation positions. The transition from the rest position in FIG. 6 to the activation position in FIG. 7 takes place when the radial protrusion 164 engaged in the cavity 211 of the contact end 210 pushes back the rod 201 of the activation element 20 counter to the force exerted by the spring 202. The transition from the activation position in FIG. 7 to the rest position in FIG. 6 takes place when the radial protrusion 164 engaged in the cavity 211 of the contact end 210 slows down and controls the movement of the rod 201 under the action of the force exerted by the spring 202. As a result, the driving element 162 drives the translational movement of the activation element 20 from its rest position to its activation position, and vice versa.

The control interface 22 comprises multiple control buttons 130, and display members 132 consisting of light-emitting diodes or LEDs, each display member being associated with a marking 134 in the form of a logo indicative of a parameter displayed by means of this member. The control interface notably enables control of the drive assembly 12 via an electronic control board 44.

The control board 44 is configured to activate the motor 42 on the basis of orders received by the interface 22 and to return the thrust element 143 of the piston 14 to its rest position when the product contained in the cartridge 10 has been used up in its entirety, or a predetermined quantity has been used up.

The elements that make up the reusable control assembly 4 are relatively elaborate and costly, hence the benefit of using them with several disposable parts 6 in succession.

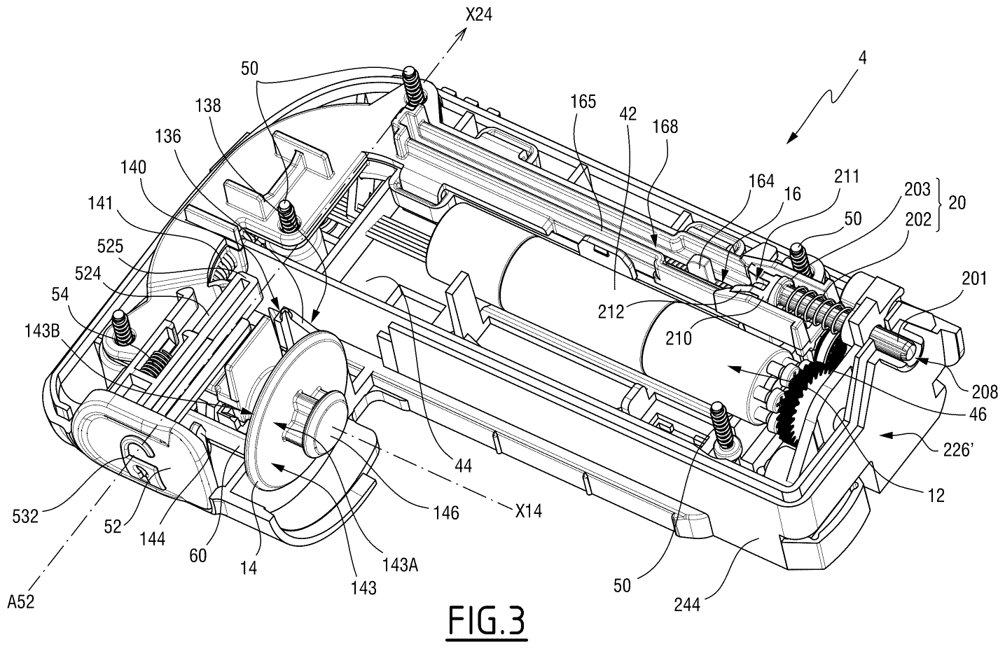

Screws 50, shown in FIGS. 3 and 4, are used to hold the half-shells 242 and 244 securely together. In the first embodiment depicted, the screws 50 pass through corresponding cavities provided in the half-shell 244 and screw into corresponding tappings in the half-shell 242. Other means of assembling the half-shells with one another are conceivable, notably elastic clip-fastening assembly means.

As is more particularly apparent from FIG. 2, the casing 8 of the disposable part 6 is equipped with a hook 88 which constitutes a securing member for holding the parts 4 and 6 together when the product administering device 2 is in the assembled configuration. The hook 88 is of one piece with the half-shell 84 and comprises a head 882 and a shank 884 connecting the head 882 to the rest of the half-shell 84.

In addition, as depicted in FIG. 3, the half-shell 244 comprises a rigid portion 136 defining a ramp 138 for the sliding of the head 882 during a movement of bringing the parts 4 and 6 closer together in the direction of the arrow F1 in FIG. 2. The rigid portion 136 also defines a catching surface 140, which is perpendicular to the axis X14 and against which the head 882 comes into abutment when the parts 4 and 6 of the product administering device 2 are in the assembled configuration.

A push-button 52 is mounted on the casing 24 and comprises a head 522, the exterior shape of which is preferably convex, received in a cavity of corresponding shape, this cavity being defined by the two half-shells 242 and 244. In the absence of action by a user on the push-button 52, the exterior surface 522S of the head 522, which is to say that surface of this head that is visible from outside the casing 24, lies flush with an exterior surface of the casing 24.

A longitudinal axis A52 of the push-button 52 is denoted A52.

The push-button 52 also comprises a rod 524 which extends along the longitudinal axis A52.

When the reusable control assembly 4 is in the mounted configuration, the axis A52 is superposed with an axis X24 which is defined by the casing 24 and is an axis of sliding of the push-button 52 in the casing 24.

By default, the push-button 52 is pushed back by two springs 54 into the position in FIGS. 1 to 3, in which its rod 524 does not interact with the hook 88 and in which the surface 522S lies flush with an outer face of the casing 24.

The external surface 522S of the head 522 bears a marking 532 depicting a padlock to serve as a reminder that the push-button 52 allows the disposable part 6 to be unlocked from the reusable control system 4. Specifically, when the head 522 is depressed into the cavity by applying to its surface 522S a force E1 directed toward the inside of the casing 24, the push-button 52 exerts, by means of an end 525 of the rod 524, a force that is oriented, and of sufficient strength, to push back the head 882 of the hook 88 so as to disengage it from the catching surface 140.

The movement of the head 882 of the hook 88 under the action of the push-button 52 is achieved as a result of flexural elastic deformation of the shank 884 of the hook 82.

Thus, the push-button 52 is translationally movable, along coincident axes A52 and X24, between a first, securing position and a second, disconnection position. In its first position, and when the parts 4 and 6 are assembled, the push-button 52 is not in contact with the hook 88, which means that it is disengaged from this hook. In other words, in this position, the push-button does not interact with the hook 88. Furthermore, in its first position, the push-button defines, with the catching surface 140, a receiving region for receiving the hook 88. In its second position, the push-button is situated in the hook-receiving region. Thus, when the parts 4 and 6 are assembled and the push-button is then moved toward its second position, the end 525 of the rod 524 of the push-button pushes the head 882 of the hook 88 back against the effect of an elastic force exerted by the rod 884 to disconnect the disposable part 6 from the reusable control system 4.

In other words, the push-button 52 allows the parts 4 and 6 of the product administering device 2 to be separated starting from the assembled configuration depicted in FIG. 1. The securing-together of the parts 4 and 6 of the product administering device 2, that is obtained by means of the hook 88, is thus reversible.

The operation of the administering device and notably of the control system according to the first embodiment is described below using FIGS. 6 to 9, which depict the control system in various configurations corresponding to various steps of operating the control system 4 for administering product to a user.

In an initial operating step, after the control system 4 has been assembled with the disposable part 6 as depicted in FIG. 2, the control system 4 is in a rest configuration depicted in FIG. 6. In the rest configuration, the thrust element 143 is positioned facing the piston plunger 104 at a distance therefrom. Furthermore, the activation element 20 is in its rest position, positioned inside the casing 24, and the driving element 162 is positioned, via the radial protrusion 164, in the cavity 211 for receiving the radial protrusion.

Then, the user positions the administering device against their skin, at the site where the injection is to take place, and fixes it by means, for example, of an adhesive patch system fixed to a face of the casing 8 that is intended to be in contact with the user's skin.

During a subsequent activation step, the user causes, by actuating for example one of the buttons 130 of the control interface 22, product to be administered. During this step, after the command to activate administration has been transmitted from one of the buttons to the control board 44, the control board 44 makes the motor 42 move the activation element 20 from its rest position to its activation position. At the end of this activation step, the control system is in its activation configuration depicted in FIG. 7. To that end, the control board 44 causes the gearset 46 to rotate in the second direction to rotationally drive the threaded rod 161 and make the driving element 162 translationally move toward the first orifice 208, in the direction of the arrow F1′ marked in FIG. 6, such that the radial protrusion 164 moves the activation element 20 and particularly the rod 201 toward the needle inserting device such that its end 209 comes into contact with the release member and permits the passage of the needle and the cannula toward the deployed position, i.e. through the user's skin. The driving element 162 exerts a force on the activation element 20 greater than that exerted by the return element 202, such that the activation element is moved from its rest position to its activation position. In the activation configuration, the rod 201 protrudes from the casing 24, particularly from the lateral face 226′, particularly through the first orifice 208 and the second orifice.

Then, during a step of bringing the piston 14, and particularly the thrust element 143, into contact with the piston plunger 104, the control board 44 causes the gearset 46 to rotate in the first direction to rotationally drive the threaded rod 161 and make the driving element 162 translationally move in a direction away from the first orifice 208, such that the driving element 162 moves in the direction of deployment of the piston 14. During this step, the driving element 162 exerts a thrust force on the piston rod 141 which transmits this force to the thrust element 143, which moves toward the piston plunger 104 and comes into contact with the piston plunger 104.

While the driving element 162 is moving toward the configuration of bringing the piston 14, and particularly the thrust element 143, into contact with the piston plunger 104, the driving element 162 tends to move away from the activation element 20. The return element 202 exerts a force on the rod 201 so as to move it toward the rest position and bring the activation element into contact with the radial protrusion 164. During this step, when the activation element is back in its rest position, the driving element 162 continues to move in the direction of deployment of the piston 14, in the direction of the arrow F1 marked in FIG. 6, and the radial protrusion 164 comes out of the cavity 211, as depicted in FIG. 8.

FIG. 8 depicts the control system in its contact configuration, obtained at the end of the contacting step, in which the thrust element 143 has been moved via the driving element 162 into contact with the piston plunger 104.

Subsequently, once the control system 4 is in its contact configuration, a discharging step is performed. During this step, the control board 44 causes the gearset 46 to rotate in the first direction so as to rotationally drive the threaded rod 161 and make the driving element 162 translationally move in a direction away from the first orifice 208, in the direction of the arrow F1 marked in FIG. 6, such that the driving element 162 moves in the direction of deployment of the piston 14 to drive the movement of the piston plunger 104, in the direction of the arrow F2 marked in FIG. 6, as far as that end where product is discharged from the cartridge 10. As a result, the product is expelled from the cartridge into the cannula and thus to the product administration site by means of the movement of the thrust element 143 and of the piston plunger 104 through the cartridge 10.

Advantageously, the driving element 162 is made to move differently between the contacting step and the discharging step. The movement of the driving element is for example continuous during the contacting step and intermittent during the discharging step.

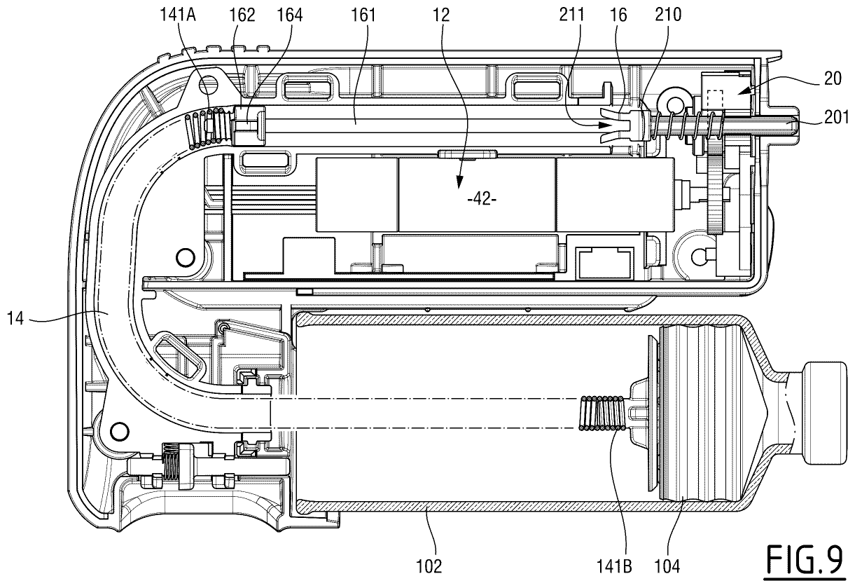

At the end of the discharging step, the administering device is back in the configuration depicted in FIG. 9, the thrust element 143 and the piston plunger 104 being close to the product discharge end of the cartridge 10 again.

Then, the control board 44 performs a retraction step in which it causes the gearset 46 to rotate in the second direction so as to rotationally drive the threaded rod 161 and make the driving element 162 translationally move in a direction toward the first orifice 208, in the direction of the arrow F1′ marked in FIG. 6, such that the driving element 162 drives the piston rod 141 and the thrust element 143 toward their retracted position, in the direction of the arrow F2′ marked in FIG. 6.

At the end of the retraction step, the control system is back in the rest configuration depicted in FIG. 6, whereas the piston plunger 104 remains at the product administering end of the cartridge 10.

Then, the parts 4 and 6 can be separated, by pressing on the push-button 52, and the part 6 disposed of. For the next use of the control system 4, a new disposable part 6 can be connected to it.

Advantageously, between the contacting and discharging steps, the control system 4 is capable of performing a step of opening a closing-off membrane for closing off the cartridge 10 that is intended to close off the product discharge end of the cartridge 10. To that end, during this step, the control board 44 causes the gearset 46 to rotate in the first direction to make the driving element 162 translationally move in the direction of deployment of the piston 14, in the direction of the arrow F1 marked in FIG. 6, in order to exert a force on the piston plunger 104 so as to increase the pressure inside the cartridge until the membrane deforms and opens up. The membrane is for example opened up by means of an opening needle positioned facing the membrane and able to pierce the membrane when it is deformed under the effect of the increasing pressure in the cartridge 10.

As a result, the administering device 2 according to the invention, and particularly the control system 4, make it possible to reliably and more simply control the administration of product and the insertion of a product administering member, such as a needle or a cannula, into a site. Furthermore, such a control system makes it possible to group together the relatively elaborate and costly constituent elements of the control assembly into a reusable assembly 4 that can be used with multiple disposable parts 6 in succession.

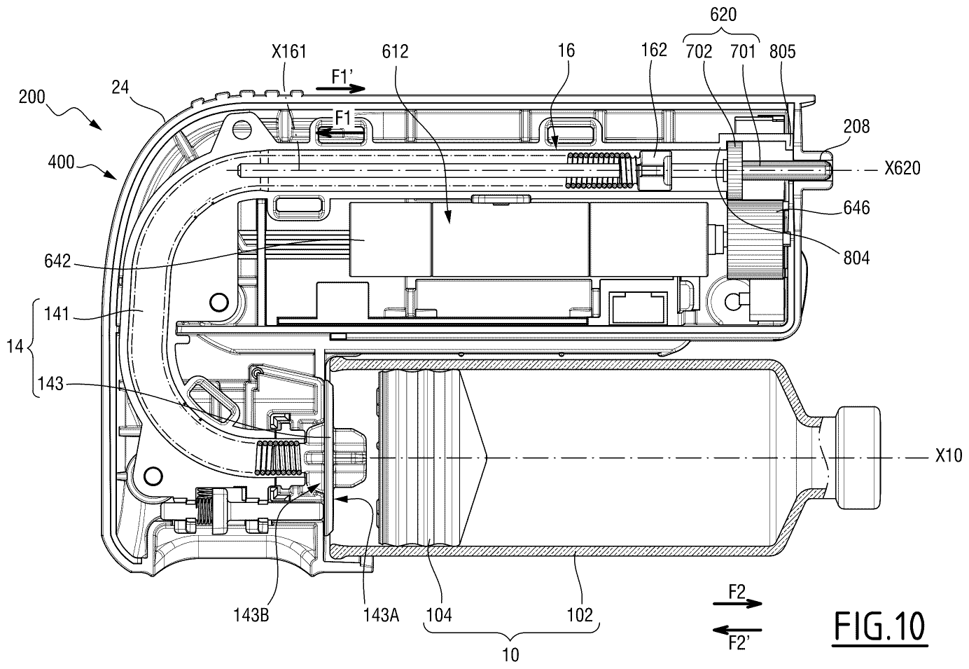

A second embodiment of a control system 400 for controlling a product administering device 200 is depicted in FIGS. 10 to 13.

In the rest of the description of the second embodiment, elements that are similar to the first embodiment have the same reference numbers and only the elements that are different are described and have different reference numbers.

More specifically, in this second embodiment, the disposable part 6 is similar to that of the first embodiment and the control system 400 comprises a drive assembly 612 and an activation element 620 which differ from the first embodiment, and also a piston 14, a control member 16 and a control interface 22 which are similar to those of the first embodiment.

The drive assembly 612 comprises an electric motor 642 and at least one toothed wheel 646 which is driven by the motor 642.

The motor 642 is able to selectively rotationally drive the toothed wheel 646 in a first direction and an opposite second direction to the first direction.

The activation element 620 comprises a rod 701 and a toothed wheel 702 which meshes with the toothed wheel 646. The rod 701 comprises for example a free activation end 709 and a contact end 710 where the toothed wheel 702 extends radially to the rod 701.

The activation element 620 is fixed to the threaded rod 161. The rod 701 is for example secured to the threaded rod 161, such that when the toothed wheel 646 is rotationally driven by the motor 642, the toothed wheel 702 is also rotationally driven, as is the threaded rod 161.

The activation element 620 is received in a receiving region Z′ comprising a first end 804 and a second end 805 which are opposite one another along an axis of movement X620 of the activation element 620. The first end 804 defines a stop in contact with the activation element 620 and particularly the toothed wheel 702, when the activation element 620 is in its rest position. The second end 805 defines a stop in contact with the activation element 620 and particularly the toothed wheel 702, when the activation element 620 is in its activation position.

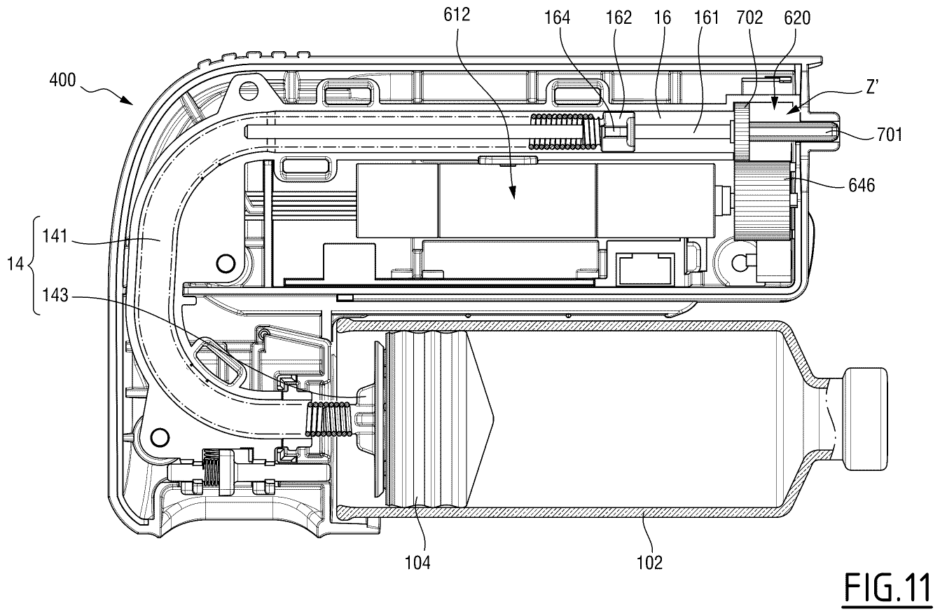

In this second embodiment, the spring which forms the flexible rod 141 is capable of exerting a thrust force on the threaded rod 161 via the driving element 162, so as to translationally move the activation element 620 from its rest position to its activation position when the thrust element 143 pushes against the piston plunger 104, as depicted in FIG. 11, and when the driving element 162 is translationally driven along the threaded rod in the direction of deployment of the piston 14.

In addition, the spring which forms the flexible rod 141 is also capable of exerting a pulling force on the threaded rod 161 via the driving element 162, so as to move the activation element 620 from its activation position to its rest position when the driving element 162 is translationally driven along the threaded rod 161 in the direction of retraction of the piston 14.

As a result, the driving element 162 drives the translational movement of the activation element 620 from its rest position to its activation position, and vice versa.

The operation of the administering device 200 and notably of the control system 400 according to the second embodiment is described below using FIGS. 10 to 13, which depict the control system 400 in various configurations corresponding to various steps of operating the control system 400 for administering product to a user. If a reference is mentioned in the following part of this description, without featuring in FIGS. 10 to 13, it refers to the same element as the one having that same reference in the first embodiment. Conversely, if a reference is featured in one of these figures without being mentioned in the description, it refers to an element similar to the one having that reference in the first embodiment.

In an initial operating step, the control system is assembled with the disposable part 6 and is in a rest configuration depicted in FIG. 10. In the rest configuration, the thrust element 143 is positioned facing the piston plunger 104 at a distance therefrom, preferably surrounded by a wall of the casing 24 that defines an opening for receiving the cartridge when the casings 24 and 8 are assembled. Furthermore, the activation element 620 is in its rest position, inside the casing 24.

Then, the user positions the administering device 200 against their skin, at the site where the injection is to take place, and fixes it by means, for example, of an adhesive patch system fixed to a face of the casing 8 that is intended to be in contact with the user's skin.

Then, the user causes, for example by actuating one of the buttons 130 of the control interface 22, product to be administered. As a result, a subsequent step of placing the piston in contact with the piston plunger is performed. During this step, the control board 44 causes the toothed wheel 646 to rotate in the direction corresponding to the direction of deployment of the piston 14. The rotation of the threaded rod 161 then makes the driving element 162 translationally move in a direction away from the first orifice 208 and the driving element 162 moves in the direction of deployment of the piston 14, in the direction of the arrow F1 marked in FIG. 10. During this step, the driving element 162 exerts a thrust force on the piston rod 141 which transmits this force to the thrust element 143, which moves toward the piston plunger 104 and comes into contact with the piston plunger 104, in the direction of the arrow F2 marked in FIG. 10.

FIG. 11 depicts the control system 400 in its contact configuration, obtained at the end of the contacting step, in which the thrust element 143 has been moved via the driving element 162 into contact with the piston plunger 104.

Subsequently, once the control system 400 is in its contact configuration, a needle inserting step is performed.

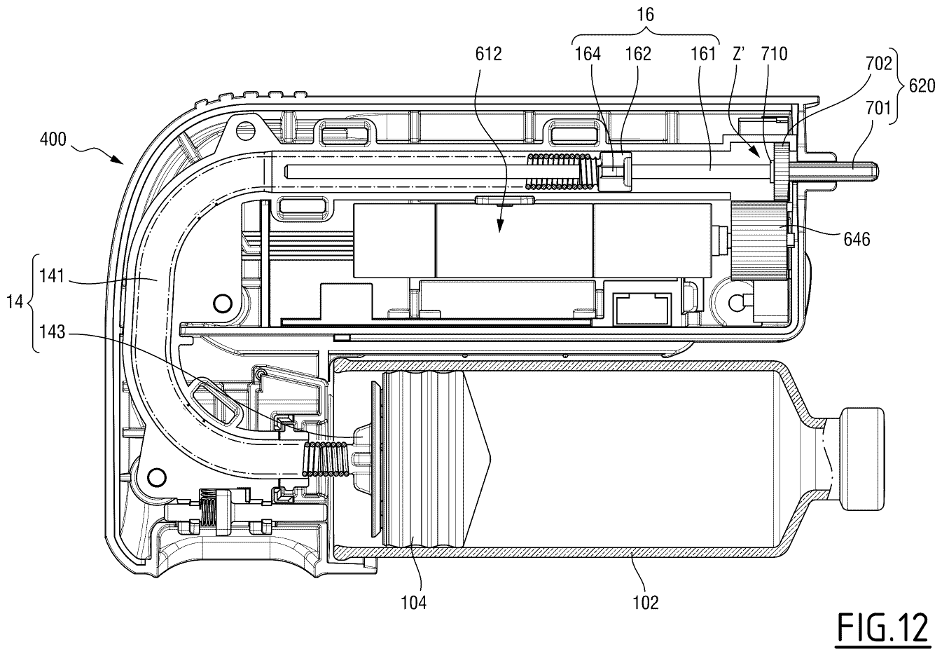

During the needle inserting step, the rotation of the toothed wheel 646 in the direction corresponding to the direction of deployment of the piston 14 continues and the driving element 162 then translationally moves in the direction of deployment of the piston 14. The driving element 162 then exerts a thrust force on the spring forming the piston rod 141, which is compressed as long as the force exerted by the piston 14 on the piston plunger 104 is smaller than the detachment force making the piston plunger 104 detach from its initial position, with respect to the cartridge 10. As a result, under the effect of the compression exerted by the driving element 162, the spring forming the piston rod 141 exerts a thrust force on the threaded rod 161 via the driving element 162, so as to move the threaded rod 161 and thus the activation element 620 from its rest position to its activation position. The control system is then in its needle inserting configuration depicted in FIG. 12, in which a free end 709 of the rod 701 protrudes from the casing 24, particularly from a surface 226′ defined as in the first embodiment.

Then, a discharging step is performed. At the start of this step, the activation element 620, and particularly the toothed wheel 702, are in contact with the second end 805, which forms a stop preventing the activation element from moving beyond the activation position. The control board 44 continues to cause the toothed wheel 646 to rotate in the direction corresponding to the direction of deployment of the piston 14. Since the activation element 620 abuts the second end 805, the force exerted by the thrust element 143 increases with the movement of the driving element 162 along the threaded rod 161 until the moment when the force exerted exceeds the force of detachment of the piston plunger 104 with respect to the cartridge 10. The piston plunger is then set in motion by the thrust element, preferably as far as that end where product is discharged from the cartridge 10.

As a result, the product is expelled from the cartridge 10 into the cannula and thus to the product administration site by means of the movement of the thrust element 143 and of the piston plunger 104 through the cartridge 10.

Advantageously, the driving element 162 is made to move differently between the contacting step and the discharging step. The movement of the driving element is, for example, continuous during the contacting step and intermittent during the discharging step.

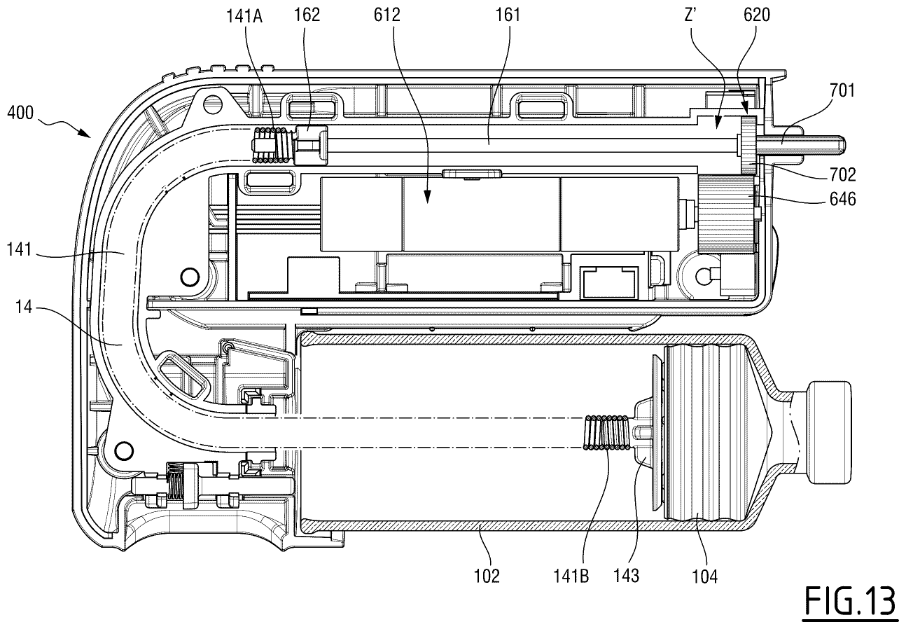

At the end of the discharging step, the administering device 200 is back in the configuration depicted in FIG. 13, the thrust element 143 and the piston plunger 104 being back at the product discharge end of the cartridge 10.

Then, the electronic board 44 performs a retraction step, in which it causes the toothed wheel 646 to rotate in a direction corresponding to the direction of retraction of the piston, the rotation of the threaded rod 161 makes the driving element 162 translationally move toward the first orifice 208 such that the driving element 162 drives the piston rod 141 and the thrust element 143 toward their retracted position.

During the retraction step, the spring forming the piston rod 141 operates in tension and no longer in compression, under the effect of the movement of the driving element 162, and is capable of exerting a pulling force on the threaded rod 161 via the driving element 162, so as to move the activation element 620 from its activation position to its rest position when the driving element 162 is translationally driven along the threaded rod in the direction of retraction of the piston.

At the end of the retraction step, the control system is back in the rest configuration depicted in FIG. 10, whereas the piston plunger 104 remains at the product discharge end of the cartridge 10. As a result, the activation element is back in its rest position, as is the thrust element.

In a variant, the control system 400 can be configured such that the translational movement, of the activation element 620 from its rest position to its activation position, is simultaneous with the thrust element 143 pushing against the piston plunger 104 of the container 10 of product.

One particular feature of the second embodiment is that the relative movement of the threaded rod 161 and of the driving element 162 is such that the driving element remains in position along the axis X620 with respect to the casing 24, whereas the threaded rod moves along that axis when it is driven by the motor assembly.

As a result, irrespective of the embodiment of the invention, the administering device 2, 200 according to the invention and particularly the control systems 4, 400 make it possible to reliably and more simply control the administration of product and the insertion of a product administering member, such as a needle or a cannula, into a site. Furthermore, the control system according to the invention makes it possible to group together the relatively elaborate and costly constituent elements of the control assembly into a reusable assembly that can be used with multiple disposable parts 6 in succession.

Irrespective of the embodiment, the driving element 162 is advantageously configured to, when it is translationally driven along the threaded rod 161, drive the activation element 20, 620 from its rest position to its activation position and from its activation position to its rest position.

Advantageously, irrespective of the embodiment, the passage of the activation element 20, 620 from its rest position to its activation position or the passage of the activation element 20, 620 from its activation position to its rest position is brought about by a translational movement along an axis X20 or X620 parallel to, in particular coinciding with, an axis of movement of the driving element 162, which in this case is the longitudinal axis X161 of the threaded rod 161. In the first embodiment, the axes X20 and X161 are parallel and offset in a direction perpendicular to the plane of FIGS. 6 to 8. In the second embodiment, the axes X620 and X161 coincide.

Advantageously, irrespective of the embodiment, the direction of movement of the activation element 20, 620 along the axis X20, X620, from its rest position to its activation position, is the same as the direction of movement of the driving element 162 along its axis of movement.

Advantageously, in its rest position and in its activation position, the activation element 20, 620, in particular its rod 201, 701, extends mainly along an axis parallel to, in particular coinciding with, the longitudinal axis X161 of the threaded rod 161.

Advantageously, the driving element 162 and the activation element 20, 620 are offset along the axis X20, X620 of movement of the activation element 20, 620.

Advantageously, the activation element 20, 620 is driven by the driving element 162 mechanically.

Advantageously, the driving element 162 is translationally moved along the threaded rod rectilinearly.

Advantageously, the activation element 20, 620 is translationally moved between its rest position and its activation position rectilinearly.

Irrespective of the embodiment considered, the product administering device according to the invention may notably be used to administer a drug in liquid form to a patient in order to treat a wide variety of conditions, such as, for example, autoimmune diseases or chronic conditions such as diabetes or the associated disorders, cancers, hormone insufficiencies, macular degeneration, inflammation, atherosclerosis, rheumatoid arthritis, coronary syndromes, thromboembolisms, etc.

Thus, the term “drug” refers to any liquid formulation that can be administered continuously by means, for example, of a hollow needle or of a cannula. The formulation may be a solution, a gel or else a fine suspension containing one or more active principles. These active principles may be notably peptides (for example, insulin or GLP-1, their derivatives or analogues, amylin or its derivatives or analogues, gastric inhibitory peptides or their analogues), proteins, glycoproteins or hormones (for example hormones derived from the pituitary gland or the hypothalamus, such as gonadotropin or gonadotropin releasing hormone, desmopressin, gonadorelin, triptorelin, terlipressin, leuprorelin, buserelin, goserelin, nafarelin, lutropin, menotropin, follicle-stimulating hormone and its analogues, follitropin, parathyroid hormone, teriparatide, abaloparatide or other parathyroid hormone analogues, calcitonin, growth hormone, somatropin, etc.), active principles derived from hormones or from nucleotides (for example DNAs, RNAs, oligonucleotides), enzymes, polysaccharides (for example glycosaminoglycans, hyaluronic acids, heparins, low-molecular-weight heparins and their derivatives, or sulfate or polysulfate forms of these polysaccharides), vaccines, antibodies and their analogues (for example denosumab, panitumumab), nutritional substances. The formulations may contain these active principles in the form of any pharmaceutically acceptable salt or solvate together with any necessary and appropriate excipient.

The embodiments and variants considered above are capable of being combined with one another to afford other embodiments of the invention.

1. A control system for controlling a product administering device, the control system comprising:
a drive assembly,
a piston,
a control member comprising:
a threaded rod intended to be rotationally driven by the drive assembly, and
a driving element translationally driven along the threaded rod when the threaded rod is rotationally driven, to cause the piston to move,
an activation element for activating a device for inserting a needle into a site, the activation element being movable between a rest position and an activation position for activating the needle inserting device,
wherein the driving element drives the movement of the activation element between its rest position and its activation position as it is translationally driven along the threaded rod,
wherein that:
the activation element is translationally movable between its rest position and its activation position.

2. The control system as claimed in claim 1, wherein the driving element drives the translational movement of the activation element from its rest position to its activation position and the translational movement of the activation element from its activation position to its rest position.

3. The control system as claimed in claim 1, wherein the piston comprises:
a flexible rod, and
a thrust element able to act on a piston plunger of a container of product to discharge the product from the container,
the flexible rod comprising a driving end intended to be in contact with the driving element and a thrust end intended to be in contact with the thrust element.

4. The control system as claimed in claim 3, wherein the flexible rod is formed by a spring and the activation element is fixed to the threaded rod, the spring being capable of exerting a thrust force on the threaded rod via the driving element, so as to move the activation element from its rest position to its activation position when the thrust element pushes against the piston plunger and when the driving element is translationally driven along the threaded rod in a direction of deployment of the piston.

5. The control system as claimed in claim 4, wherein the driving end is fixed to the driving element and the thrust end is fixed to the thrust element, and wherein the spring is capable of exerting a pulling force on the threaded rod via the driving element, so as to move the activation element from its activation position to its rest position when the driving element is translationally driven along the threaded rod in a direction of retraction of the piston.

6. The control system as claimed in claim 1, wherein the driving element is capable of:
moving the piston to cause the product to be discharged from the container, when the driving element is translationally driven along the threaded rod in a direction of deployment of the piston,
moving the activation element from its rest position to its activation position, when the driving element is translationally driven along the threaded rod in a direction of retraction of the piston.

7. The control system as claimed in claim 1, wherein the system comprises a return element capable of exerting a return force on the activation element for returning it to its rest position.

8. The control system as claimed in claim 7, wherein the activation element comprises a rod around which extends the return element and a shoulder in contact with the return element.

9. The control system as claimed in claim 1, wherein the activation element is received in a region comprising a first end and a second end which are opposite one another along an axis of movement of the activation element, the first end defining a stop in contact with the activation element when it is in its rest position.

10. The control system as claimed in claim 9, wherein the second end defines:
either a second stop in contact with the activation element, when it is in its activation position,
or a bearing surface for a return element.

11. The control system as claimed in a claim 1, wherein the system comprises a first casing for receiving the drive assembly, the control member and the activation element, and wherein the first casing comprises a first orifice for the passage of the activation element and through which the activation element can pass when it is in its activation position, the activation element then protruding from the first casing.

12. The control system as claimed in claim 1 wherein the system comprises a first casing for receiving the drive assembly, the control member and the activation element, and wherein the first casing is capable of being assembled detachably with a second casing which receives the needle inserting device and advantageously a container of product.

13. The control system as claimed in claim 3, wherein the system comprises a first cavity capable of receiving the flexible rod, the threaded rod and the driving element, the first cavity comprising a wall provided with a longitudinal slot, and wherein the driving element comprises a radial protrusion intended to be received in the longitudinal slot, so as to prevent the driving element from rotating relative to the first cavity.

14. The control system as claimed in claim 13, wherein the activation element comprises a contact end and an activation end, the contact end defining a cavity for receiving the radial protrusion, when the driving element drives the activation element from its rest position to its activation position via the radial protrusion.

15. The product administering device comprising a first part provided with a control system as claimed in claim 1 and a second part which receives the needle inserting device and a container of product.

16. The device as claimed in claim 15, wherein the control system and the second part comprises a second casing for receiving the needle inserting device, the second casing being intended to be assembled detachably with the first casing, and wherein the second casing comprises a wall provided with a second orifice capable of being positioned facing the first orifice when the first casing and the second casing are assembled, the activation element being capable, when it is in its activation position, of passing through the second orifice to activate the needle inserting device and trigger the insertion of the needle into a site.
